(19) United States
(12) Patent Application Publication
(10) Pub. No.: US 2010/0143475 A1
(43) Pub. Date: Jun. 10, 2010

(54) TRANSDERMAL THERAPEUTIC SYSTEM WITH TWO-PHASE RELEASE PROFILE

(76) Inventors: Johannes Tack, Berlin (DE); Björn Schurad, Munchen (DE); Antje Müller-Schubert, Berlin (DE); Reinhard Horowski, Berlin (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/311,305

(22) PCT Filed: Aug. 27, 2007
(86) PCT No.: PCT/EP2007/058867

§ 371 Date: Jan. 20, 2010

(30) Foreign Application Priority Data
Oct. 6, 2006 (DE) 10 2006 048 130.5

(51) Int. Cl. A61K 31/48; A61K 9/00; A61P 25/16; A61P 25/14; A61P 25/00

(52) U.S. Cl. 424/484; 514/288

(57) ABSTRACT

The present invention relates to a transdermal therapeutic system (TTS) consisting of an impermeable coating, a matrix containing an ergoline compound having the formula (I)

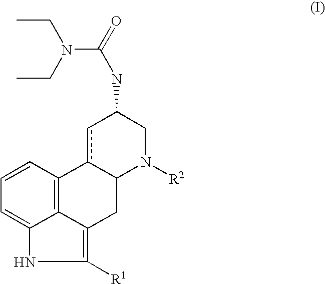

or a physiologically compatible salt or derivative thereof, wherein R¹ denotes an H atom or a halogen atom and R² is an alkyl group having 1 to 4 carbon atoms and [symbol] denotes a single or double bond, and a removable protective layer, wherein the ergoline compound or a physiologically compatible salt or derivative thereof is stabilised by an antioxidant and a basic polymer. The TTS is characterised in that the matrix contains at least one hydrocarbon having 8 to 18 carbon atoms in a straight or branched chain, which has a functional group at the end of the alkyl chain and/or Aloe Vera, so that in a first phase (0-5 hours after application) only 0-20% of the therapeutically desired steady-state plasma concentration of the ergoline compound is achieved and the therapeutically desired steady-state plasma concentration of the ergoline compound is only achieved in a second phase (5-20 hours after application).

The present invention relates to a transdermal therapeutic system for ergoline compounds having a new two-phase release profile, in which in a first phase (0-5 hours after application) only 0-20% of the therapeutically desired steady-state plasma concentration of the ergoline compound is achieved and then the therapeutically desired steady-state plasma concentration of the ergoline compound is only achieved in a second phase (5-20 hours after application).

Present-day dopaminergic therapies for Parkinson's disease and other states such as restless legs syndrome and other neurological diseases which are associated with brain damage or brain injuries are adversely affected by a large number of side effects, if they are completely effective. The adjustment of oral dopaminergic therapies is made either using levodopa alone or with the aid of boosters (MAO inhibitors, COMT inhibitors) or dopamine agonists. At the same time, one complies with the individual bioavailability and the therapeutic needs of a patient, i.e. the therapy is, so to speak, titrated-in. This titration can comprise up to five different active substances with unpredictable metabolism or other interactions. It is based on the induction of side effects such as nausea, vomiting, fatigue, dizziness, orthostatis, but likewise dyskinesia or mental impairments. These side effects are used as indicators for the bioavailability of these active substances. Following the incidence of these effects, the dose is subsequently reduced and many attempts are made to build up a tolerance to these effects in order to prevent them or subsequently keep them at a low level.

Other approaches involve dividing the effective daily dose into several administrations or the combination of these active substances with other active substances in order to reduce the side effects. For example, peripheral decarboxylase inhibitors are used, this being an essential prerequisite in the case of levodopa therapy. In the case of dopamine agonists or clozapine etc., domperidone, for example, is used. The build-up of an appropriately effective and, to some extent, well-tolerated dopaminergic treatment requires 6 to 12 weeks under constant medical monitoring. It is dependent on the availability of a large number of different dosage forms and fast- or slow-release preparations.

The available data from comprehensive clinical studies which are published in the literature are presented in Table 1.

In this context, the abbreviations of the references denote the following specified citations:
Rinne, 1983 Rinne, K.; New ergot derivatives in the treatment of Parkinson's disease; in: eds. Caine, D. B., Horowski, R., McDonald, R. J., Wuttke, W.; Lisuride and other dopamine agonists; Raven Press New York, (1983), 431-442
Quinn, 2002 Quinn, N.; A multicenter, double-blind, randomized, placebo-controlled safety and efficacy study of rotigotine constant delivery system (CDS) in patients with advanced Parkinson's disease; Poster presented at the 14th International Congress on Parkinson's Disease, Helsinki 2001
PDR 58, 2004 Physicians' Desk Reference 58, 2004, Thomson PDR at Montvale, N.J. 07645-1742

TABLE 1
Peripheral side effects (nausea, vomiting) following application of dopamine agonists

| | -1- | -2- | -3- | -4- | -5- | -6- | -7- |
|---|---|---|---|---|---|---|---|
| Active substance | Bromocriptine | Lisuride | Rotigotine | Cabergoline | Pergolide | Ropinirol | Pramipexol |
| | | | CDS | | | | |
| Study | Double blind | Open | Double | Double | Double | Double | Double |
| | | | blind | blind | blind | blind | blind |
| Active treatment | 231 | 29 | 171 | 221 | 189 | 157 | 388 |
| (n =) | | | | | | | |
| Mode of application | oral | oral | transdermal | oral | oral | oral | oral |
| Dose range | 5.0-30.0 mg | 0.6-4.0 mg | 4.5-18 mg | 0.25-1.0 mg | 0.25-1.0 mg | 0.5-5.0 mg | 0.375-4.5 mg |
| GI side effects: | | | | | | | |
| Nausea/vomiting | 43 | 37.9 | 45.6 | 29.0 | 27., 0 | 45.8 | 12.4 |
| (%) | | | | | | | |
| References | PDR 58, 2004 | Rinne, | Quinn, 2002 | PDR 58, | PDR 58, | PDR 58, | PDR 58, |
| | | 1983 | | 2004 | 2004 | 2004 | 2004 |

These data demonstrate that the known preparations of dopamine agonists cause a high degree of peripheral side effects such as nausea and vomiting, in particular in the high-titration phase.

A transdermal preparation of a dopamine agonist (Table 1; 3-) which is normally produced to continuously release the active constituent is also clearly not generally suitable for significantly reducing the peripheral side effects compared to conventional immediate-release preparations.

Some indications can be found in the literature that transdermal preparations can compensate for skin-binding effects of the active substance during the first phase of the release by overloading the system and/or introducing defined quantities of the active substance into the outer adhesive layer. This probably indicates a more constant release of the active substance to the systemic circulation but in the case of active substances having a narrow therapeutic range, is an unsuitable method of administration to prevent side effects.

Variations in the therapeutic combination are frequently necessary for a plurality of reasons. In such cases, patients must quite frequently remain in special clinics or similar facilities, often for one month or longer. It is quite frequently the case that patients with advanced diseases are treated with up to three different preparations of levodopa of different strength, one or two dopamine boosters and additionally one or two dopamine agonists (one short-term acting agonist for providing efficacy peaks, one long-term acting agonist to cover the night). In addition, one or two additional active substances are added to reduce side effects of this combination. This results in an intake frequency of six or more times a day. Furthermore, an injectable active substance is frequently added for cases of emergency. This results in the need to tune all the activities of everyday life, which is already severely compromised by the disease, to the therapy. Otherwise, it would not be possible to follow such complex schedules. All this affects a fragile, multimorbid elderly population which frequently suffers not only from reduced motor function but frequently also from varying alertness (vigilance) and cognitive impairment. These problems are exacerbated with advanced disease. The patients also develop problems with swallowing which not infrequently leads to shock or ultimately to aspiration pneumonia. Patients with impaired cognition or impaired consciousness could also benefit from dopaminergic therapies which can improve the consciousness, motor functions, conditions and also neurodegeneration. These impairments can result from direct damage to the brain, where the cause can either be traumatic brain injuries, poisoning, vascular damage or many other factors. For the said reasons, however, a dopaminergic oral therapy is not the suitable method in all cases although it could theoretically be helpful.

It is obvious that in this situation, parenteral therapies, for example using apomorphine or lisuride, have been studied and have in fact shown a higher efficacy. This applied, for example, to intravenous, subcutaneous or intraduodenal infusion to achieve a continuous dopaminergic stimulation. However, this has so far only taken place in very selected groups of patients since the side effects can also be serious and occur frequently and the symptoms are burdensome. Thus, injections, for example, of apomorphine using a penject system have only been approved for emergency therapy of severe Parkinson's syndrome akinesia. The severe nausea-inducing effect of apomorphine and also of lisuride, and other side effects have also prevented these being used in patients with advanced Parkinson's disease since this can in turn easily lead to aspiration and pneumonias or to circulatory collapse. As a result of this, these applications were completely excluded in states of reduced or lost consciousness. Attempts to reduce this nausea-inducing and or thostatic effect by administering domperidone or other active substances failed since these active substance require oral administration which in most cases in not feasibly with these patient groups.

Consequently, there is a need for further pharmaceutical forms of dopamine agonists which exhibit a lower degree of peripheral side effects. These should be superior to the previously known preparations.

It is therefore the object of the present invention to provide a transdermal therapeutic system for the administration of dopamine agonists which shows significantly reduced side effects compared with the previously known padministration forms.

The object is achieved by a transdermal therapeutic system (TTS) according to claim 1. Further preferred embodiment are obtained from the dependent claims.

In other words, the object is achieved by a transdermal therapeutic system (TTS) consisting of an impermeable coating, a matrix containing an ergoline compound having the formula (I)

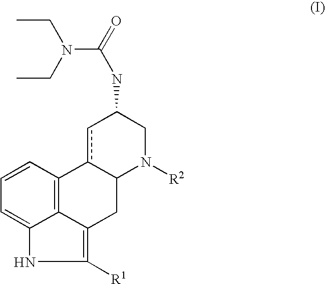

or a physiologically compatible salt or derivative thereof, wherein R¹ denotes an H atom or a halogen atom and R² is an alkyl group having 1 to 4 carbon atoms and [symbol] denotes a single or double bond, and a removable protective layer, wherein the ergoline compound or a physiologically compatible salt or derivative thereof is stabilised by an antioxidant and a basic polymer, wherein the TTS is characterised in that the matrix contains at least one hydrocarbon having 8 to 18 carbon atoms in a straight or branched chain, which has a functional group at the end of the alkyl chain and/or Aloe Vera.

The TTS according to the invention is further characterised in that in a first phase (0-5 hours after application) only 0-20% of the therapeutically desired steady-state plasma concentration of the ergoline compound is achieved and the therapeutically desired steady-state plasma concentration of the ergoline compound is only achieved in a second phase (5-20 hours after application).

The term steady-state plasma concentration describes the concentration of lisuride in the blood plasma at which the resorbed quantity of the active substance is equal to the eliminated quantity so that a constant plasma concentration is achieved over time.

The blood samples taken over the duration of application of the TTS plaster were converted into blood plasma and the lisuride content was determined by means of selective analytical methods (RIA or LC/MS/MS). By plotting the plasma concentrations of lisuride thus determined vs. the time, the steady-state plasma concentration could be determined from the plateau-like course of the profile.

For lisuride as an example of an ergoline compound according to the invention or a physiologically compatible salt thereof, the desired plasma concentration is 5 pg lisuride per ml to 10 ng lisuride per ml, preferably 50 to 500 pg lisuride per ml. A steady-state plasma concentration of 100 to 200 pg lisuride per ml is most preferred. All the concentration details refer to the quantity of lisuride per ml blood plasma volume.

In the case of a different ergoline compound in the sense of the present invention, the preferred plasma concentration is determined according to the active potency of the compound.

The transdermal therapeutic system (TTS) according to the invention is further characterised in that the at least one hydrocarbon having 8 to 18 carbon atoms in a straight or branched chain preferably has a hydroxyl or amino group or a pyrrolidone ring or an —OOCCH₂N(CH₃)₂ group as the functional group at the end of the alkyl group. Particularly preferably, the at least one hydrocarbon having 8 to 18 carbon atoms in a straight or branched chain has a hydroxyl group (alcohol) as the functional group at the end of the alkyl group.

According to the invention, it is preferable for the trans-dermal therapeutic system that the at least one hydrocarbon having a functional group at the end of the alkyl chain has 10 to 14 carbon atoms in a straight or branched chain.

Particularly preferably, the at least one hydrocarbon having a functional group at the end of the alkyl chain has 12 carbon atoms in a straight or branched chain.

In a most preferred embodiment, the at least one hydrocarbon having a functional group at the end of the alkyl chain is 1-dodecanol.

The at least one hydrocarbon having a functional group at the end of the alkyl chain has a content of 0.001 to 20.00 wt. % in the transdermal therapeutic system according to the invention. The content is preferably 0.50 to 15.00 wt. %, the content in a very preferred embodiment being 1.00 to 10.00 wt. %. Most preferably, the at least one hydrocarbon having a functional group at the end of the alkyl chain has a content of 10.00 wt. %.

The transdermal therapeutic system according to the invention is further characterised in that the Aloe Vera oil contained in the matrix was obtained from a vegetable oil, preferably peanut oil, almond oil, sesame oil or soya oil.

The Aloe Vera oil is particularly preferably obtained from soya oil. The extraction was carried out from the fresh leaves of the plant.

The content of Aloe Vera oil in the transdermal therapeutic system according to the invention is 0.01 to 20.00 wt. %. The content of Aloe Vera oil is preferably 0.5 to 10.00 wt. %. In a most preferred embodiment, the transdermal therapeutic system contains 5.00 wt. % of Aloe Vera oil. %.

The ergoline compound contained in the transdermal therapeutic system according to the invention is preferably lisuride or proterguride or a physiologically compatible salt or derivative thereof. A particularly preferred embodiment of the transdermal therapeutic system contains lisuride (cf. Formula II) or proterguride (cf. Formula III) as the ergoline compound.

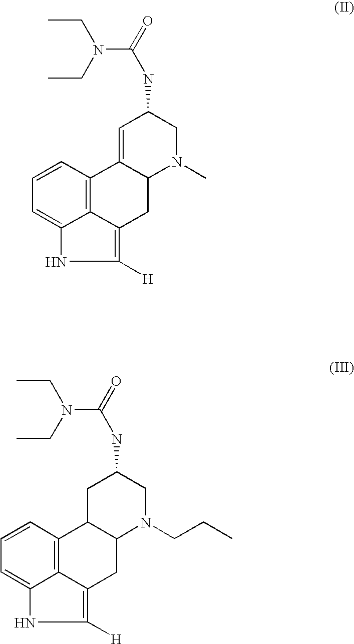

According to the invention, the content of the ergoline compound or the physiologically compatible salt or derivative thereof is 0.50 to 20.00 wt. % in the matrix of the transdermal therapeutic system. The transdermal therapeutic system preferably has a content of the ergoline compound or the physiologically compatible salt or derivative thereof of 3.00 to 6.00 wt. %.

In addition to the features already specified, the TTS according to the invention is also characterised in that the matrix can contain penetration-boosting means.

In a preferred embodiment, the matrix can have a covering diffusion barrier and an adhesive layer which is permeable to the substances of formula (I).

The antioxidant contained in the TTS according to the invention is preferably selected from the group of di-tert. butyl methyl phenols, Cert. butyl methoxyphenols, tocopherols and/or ubiquinones. The antioxidant is preferably present in quantities of 0.25 wt. % to 5.00 wt. %.

The basic polymer contained in the TTS according to the invention is preferably an acrylate (co)polymer, a butylmethacrylate-(2-diaminoethyl)methacrylate-methacrylate copolymer being particularly preferred.

According to the invention, the basic polymer can be contained in the matrix or the adhesive layer.

The basic polymer preferably contains an adhesive force booster in the matrix or the adhesive layer.

This adhesive force booster preferably contains resins (modified or unmodified) and/or neutral polyacrylates. In a particularly preferred embodiment, the TTS according to the invention contains 1 to 20 wt. % of adhesive force booster, a content of 2 to 10 wt. % of adhesive force booster being most preferred.

By means of detailed studies, it has been shown that when using a transdermal system without Aloe Vera oil and without at least one hydrocarbon having 8 to 18 carbon atoms in a straight or branched chain, having a functional group at the end of the alkyl chain, merely a single-phase release mode is achieved.

In contrast, the additional introduction of at least one hydrocarbon having 8 to 18 atoms in a straight or branched chain, having a functional group at the end of the alkyl chain, resulted in a significant delay in the onset of steady-state active-substance diffusion. In the second phase between 5 and 48 hours, significantly increased transdermal flow values were obtained for the active substance, thereby delineating a two-phase release profile. A corresponding two-phase profile was also obtained by adding Aloe Vera oil (without a representative of the aforementioned hydrocarbons) to the preparation. Joint introduction of Aloe Vera oil and at least one hydrocarbon having 8 to 18 atoms in a straight or branched chain, having a functional group at the end of the alkyl chain, additionally results in a two-phase release profile in a significantly more defined form. This can be identified from a further time delay of the onset of the steady-state flow.

By means of the TTS' according to the invention as described above, a new method has been found for patient-friendly administration of dopamine agonists. Application produces a continuous dopaminergic stimulation at a relatively low active substance plasma concentration but whilst maintaining a constantly high efficacy which commences rapidly. The need for adjustment by titration on the basis of side effects is eliminated. As a result of the two-phase release, the tolerability is improved substantially since side effects such as vomiting and nausea can be reduced significantly. A substantially improved risk-benefit profile also results.

By means of placebo-controlled double-blind trials on 335 Parkinsonism patients, it was confirmed that most or all the usual side effects which occur very frequently with all the other dopamine-like therapeutic treatments and active substances can be avoided (nausea, vomiting, orthostasis, dizziness). In addition, pharmacokinetic data which were obtained from further clinical trials reveal a plasma concentration profile of the active substance in which no sharp peak levels occur. At the same time, under repeated administration, transition levels are maintained in the pharmacologically effective concentration range where a favourable ratio of peak to transition concentration of around four is present.

The use of the TTS according to the invention thus provides specific and well-defined two-phase release profiles of the dopaminergic stimulant. Surprisingly, long drawn-out, high titration can thus be circumvented and side effects avoided without pre-treatment or concomitant active substances. At the same time, a very strong therapeutic effect can be achieved, which commences within the first few days of the therapy. This effect can also be achieved in situations in which impaired cognition and/or severely impaired consciousness is present. Furthermore, in most cases there is no further need for combinations of active substances. The treatment is gentle and patient-friendly so that the compliance of the patients and their quality of life are substantially improved.

Due to the favourable two-phase release profile, the TTS according to the invention is suitable for the treatment of neurodegenerative diseases, in particular Parkinson's disease and Parkinsonism (Parkinson syndrome). Furthermore, the TTS according to the invention is suitable for the treatment of restless legs syndrome and for the treatment of other neurological damage accompanying brain damage and brain injuries.

The present invention is explained hereinafter with reference to examples.

### EXAMPLES

### Example 1

In vitro skin permeation of a lisuride-containing transdermal therapeutic system (TTS) using skin of a human cadaver (or excised skin of hairless mice)

Lisuride was dissolved in organic solvents and mixed with a pressure-sensitive polyacrylate adhesive, dodecanol, Aloe extract, polyvinylpyrrolidone, butylhydroxytoluene and—if necessary—with further adjuvants to modify the physical properties of the resulting laminate. The mixture was applied to a fluoropolymerised release liner and dried to completely remove the organic solvent before being laminated with a polyethylene (PE) back membrane. The lisuride content was 5% and the coating weight of the dry adhesive coating was determined as 5 mg/cm². Circular samples having a diameter of 1.2 cm were punched from this laminate by means of Henkel hollow punches and were adhesively bonded to the stratum corneum of the suitably prepared skin segments after removing the release liner. The skin segments thus prepared were now inserted in classical, static diffusion cells so that the underside of the skin was in direct contact with the acceptor medium used. A modified pH 7.4 phosphate-buffered solution having sufficient solubility for the active substance to ensure “sink” conditions during the entire experiment functioned as the acceptor medium. The medium was permanently temperature-controlled at 38° C. which resulted in a temperature of 32° C. in the diffusion range.

At predetermined times, a sample of the acceptor medium was taken and investigated for its lisuride content by means of specific chromatographic methods. The corresponding volume was replaced by fresh pre-heated medium, the dilution being included in the calculations when determining the amount of active substance which has penetrated. The total amount released from the adhesive coating (n≧3) was plotted as a function of the time duration of the diffusion experiment. The corresponding figures (FIGS. 1a to 1c) show that in the absence of the chemical permeation boosters/modifiers, 1-dodecanols and Aloe Vera oil, a single-phase release profile could be observed which exhibited no significant time delay of the active substance diffusion (FIG. 1a). This was confirmed by the steady-state regression line which has a point of intersection with the X axis of approximately zero. In contrast, the additional introduction of 1-dodecanol into the matrix of the TTS resulted in a delay in the onset of steady-state active substance diffusion, whereby a two-phase release profile was achieved. Furthermore, significantly increased transdermal flow values were obtained for the active substance in the second phase between 5 and 48 hours (FIG. 1b). This two-phase release profile was also obtained when adding Aloe Vera oil to the preparation and even more so, when 1-dodecanol and Aloe Vera oil were introduced jointly (FIG. 1c).

### Example 2

### Application of a Lisuride-Containing TTS to Humans

In a clinical trial, seven elderly probands received a lisuride-containing TTS of up to 30 cm². The TTS had an identical composition and was produced under the same process conditions as the preparation from Example 1. The lisuride content was 0.25 mg/cm² and the weight of the dry coating was 5 mg/cm². The application time was 48 hours, then the plasters were removed and investigated for their remaining content of active pharmaceutical constituent. Blood samples were taken at predetermined times and converted into plasma. The lisuride concentration in each plasma sample was measured by means of a specific radio immunoassay (LLoQ=50 pg/ml; Lit. Hümpel, M., Nieuweboer, B., Hasan, S. H., Wendt, H.; Radioimmunoassay of plasma lisuride in man following intravenous and oral administration of lisuride hydrogenmaleate; Effect on plasma prolactin level; Eur. J. Clin. Pharmacol. 20, (1981), 47-51). The measured concentrations were plotted as a function of time for each application. The resulting curve shows a two-phase profile, wherein in the first few hours (about 0 to 3 hours) no permeation of the active substance from the plaster through the skin into the blood stream takes place (or only in non-quantifiable, negligible quantities). In the following hours (3-8(10) hours), the transdermal throughput of the medicinal substance increases. A second absorption phase then follows with a significantly higher rate. This has the result that the plateau is reached after about 12 hours, this plateau reflecting the maximum and in particular the desired plasma concentration for the therapy. Overall, a continuous release of the active substance from the applied TTS is assumed (cf. FIG. 2).

### Example 3

### Simulation of Repeated Application of Lisuride TTS to Humans

The plasma concentration—time profile after transdermal administration of a lisuride TTS for 48 hours was taken as a data base to mathematically simulate the repeated application of the said lisuride plaster in accordance with the superimposition principle. FIG. 3 shows the plasma concentration profile after the simulation of four successive applications of a 40 cm² lisuride TTS. The profile shows no cumulation and no sharp peak concentrations. Furthermore, it exhibits clinically effective concentrations at the times of the transition level. The simulation reveals a clinically useful ratio of peak to transition level of the lisuride of no more than three to five, which assists in the avoidance of peak dose dyskinesias and likewise of transition level akinesias in a therapy, in particular of advanced Parkinson's disease.

### Example 4

### Multiple Dose Pharmacokinetics in 18 Probands with Restless Legs Syndrome

In an open clinical trial, a 20 cm² lisuride TTS was administered for 168 hours repeatedly to 18 probands having restless legs syndrome (18 to 65 years of age, BMI of 18 to 38 kg/m²). The application site was the upper arm and changed from one arm to the other between two successive periods. The lisuride concentrations in plasma samples were measured at predetermined times by means of a specific LCMS/MS assay, LLoQ=10 pg/ml. The results are given in Table 2.

TABLE 2
Pharmacokinetic parameters of lisuride after repeated
application of a 20 cm² TTS.

| | | Transition | Ratio of |
|---|---|---|---|
| | Peak | level | peak/transition |
| | level | (pg/ml) | level (pg/ml) |
| First application | 120 | 32 | 3.7 |
| Second application | 125 | 23 | 5.4 |
| Third application | 130 | 31 | 4.2 |
| Fourth application | 140 | 32 | 4.4 |

### Example 5

### Clinical Trials with a Transdermal Lisuride TTS in Patients with Advanced Parkinsonism

The double-blind randomised clinical trial included patients with advanced Parkinson dyskinesia (PD) for which only unsatisfactory therapeutic success had been achieved with oral therapies (PD), as is demonstrated by 2 hours “off” per day or a total of ≧6 hours “off” within the last three days. The patients were trained to assess their state themselves. An improvement in their time “offs” compared with the base line is the primary efficacy end point. Second efficacy end points are UPDRS (motor part and activities of daily life (ADL), alone or in combination) and general measurements (determination by physicians and patients, CGI, QoL scale). Disadvantageous side effects were registered in the usual way. In a concomitant anti-PD therapy, oral dopamine agonists were not permitted and the dosage of all other anti-PD active substances had to be stable for at least four weeks before the trial. 50% of the patients were administered the lisuride TTS and the other half were administered an identical placebo plaster. The data are presented in Tables 3 and 4 and in FIG. 4.

TABLE 3
Trial population at baseline (BL)

| | Lisuride | Placebo |
|---|---|---|
| n patients (FAS) | 168 | 165 |
| Age | 64.2 ± 8 | 64.5 ± 9 |
| Sex % | 60.7% | 52.7% |
| H + Y stage I | 2 | 6 |
| H + Y stage II | 138 | 123 |
| H + Y stage III | 27 | 35 |
| H + Y stage IV | 1 | 1 |
| Time since | | |
| PD diagnosis (M) | 107.5 | 103.6 |
| L-DOPA (M) | 87 | 83 |
| With dyskinesias | 56.5 | 52.6 |
| UPDRS II + III | 39.3 | 40.9 |
| Total “off” hours | 5.72 | 5.85 |

TABLE 4
Peripheral side effects following transdermal application
of a lisuride TTS in 335 Parkinsonism patients

| | Lisuride | Placebo |
|---|---|---|
| | TTS | plaster |
| Nausea, vomiting, orthostasis | 4.2% | 5.4% |
| Drowsiness (mild) | 3.0% | 1.8% |
| Hallucinations | 5.4% | 1.8% |
| total psychiatric AE's | 12.5% | 6.6% |
| Dyskinesias | 7.1% | 3.0% |

Since the total daily “off” time decreases significantly, the total “ON” time without troublesome dyskinesias shows a corresponding increase. However, no increase in the dyskinesias can be registered, as was expected in the case of increasing daily levodopa dosage and frequency or with levodopa boosters (MAO-B or COMT inhibitors).

When compared with all oral DA agonist trials, it is striking that there is no difference in the frequency of incidence of gastrointestinal converse effects. This confirms the concept that these peripheral side effects are caused by rapidly increasing active substance levels in the blood (and therefore at the CTZ which is localised outside the blood-brain barrier). In the lisuride group there were five cases of nausea (3%) which were considered to be related to the active substance (plus one case of nausea and one case of vomiting which were not considered to be related to the active substance, i.e. 4.2% in total). In the placebo group six cases were considered to be related to the active substance (3.6%) and one case of vomiting (0.6%) whereas another case (0.6%) and one case of nausea (0.6%) were considered to be not related to the active substance. It is not clear whether the high number of psychiatric reactions and dyskinesias in the lisuride group is based on chance or whether it indicates that the upper end of the therapeutic range had already been reached in some patients. Answers to these questions could be provided by an individual titration under “real life” conditions.

TABLE 5
Comparison of central side effects of transdermal lisuride following application to
335 Parkinsonism patients compared with other oral dopamine agonists

| | Duration of trial | | | | | | | | |
|---|---|---|---|---|---|---|---|---|---|
| | | | | 7 weeks dose | | | 14 weeks, | | |
| | 6 months, oral | | | esc. +4 weeks, oral | | | transdermal | | |
| | Daily dose | | | | | | | | |
| | Up to 24 mg | | | 4.5 mg or max. | | | | | |
| | (8 mg TID) | | | tol. dose | | | 2 (20 cm²) QOD | | |
| | Ropinirole | Placebo | Δ | Pramipexol | Placebo | Δ | Lisuride | Placebo | Δ |
| Hypotension | 2 | 1 | 1 | 8.8 | 2.3 | 6.5 | 2.4 | 1.2 | 1.2 |
| Syncope | 3 | 2 | 1 | — | — | — | 0 | 0 | 0 |
| Dizziness | 26 | 16 | 10 | 2.9 | 2.7 | 0.2 | 0.6 | 3 | 0^{♡} |
| Drownsiness | 20 | 8 | 12 | 9 | 7 | 2 | 3.6 | 1.8 | 1.8 |
| Fatigue | 8^{♦} | 4^{♦} | — | 29.4 | 4.5 | 24.9 | 1 | 0 | 1 |
| Dyskinesias | 34 | 13 | 21 | 14.7 | 4.5 | 10.2 | 7.1 | 3 | 4.1 |
| Nausea | 30 | 18 | 12 | 8.8 | 6.8 | 2 | 4.2 | 3.6 | 0.6 |
| Vomiting | 7 | 4 | 3 | — | — | — | 1.2 | 0.6 | 0.6 |
| Hallucinations | 10 | 4 | 6 | 5.9 [symbol] | 0 [symbol] | 5.9 [symbol] | 5.4 | 1.8 | 3.6 |
| Confusion | 9 | 2 | 7 | 10 | 7 | 3 | 1.2 | 0 | 1.2 |
| Other | 19 | 9 | 10 | 28.3 | 15.9 | 12.4 | 8 | 9 | 0^{♡} |
| psych.* | | | | | | | | | |
| Skin reactions | NA | NA | NA | NA | NA | NA | 28 [symbol] | 4.2 [symbol] | 23.8 [symbol] |

— = no information.
^{♡}= less than placebo
^{♦}= Data from restless legs study. [symbol]
= in the 8 month trial 17% of patients who received pramipexol reported hallucinations compared with 4% in the placebo group (Δ = 13).
*other psych. = amnesia, fear, abnormal dreams, nervousness, delusions, paranoid reactions, sleeplessness. [symbol]
= Skin reactions resulting in discontinuation: 12.5% in the lisuride group; 1.2% in the placebo group (Δ = 11.3).

### Example 6

### Peripheral Side Effects Following Oral Application of Lisuride in Parkinsonism Patients

20 patients with Parkinson's disease (age 19 to 73; average age 53) were combined in a double blind comparison within the patients of oral lisuride and placebo. The lisuride dose was increased up to the maximum tolerance dose of 5 mg daily using a conventional immediate-release tablet preparation of lisuride (in vitro release of more than 80% of the declared dose within 30 minutes, see FIG. 5). The dosage of other active substances was kept unchanged during the trial. Lisuride was given additionally to the already existing therapy, Converse reactions were evaluated by a physician who was not involved. In addition to the efficacy results of the treatment, gastrointestinal side effects in particular were observed. Gastrointestinal symptoms, in particular nausea, were found in eight patients (40%).

### Example 7

### Production of a Lisuride TTS with a Two-Phase Release Profile

Succinic acid (5%) was dissolved in a mixture of acetone and 2-propanol, Eudragit E 100 polymer (43%) was slowly added and dissolved while stirring, dibutylsebacate (19%) was slowly added and dissolved while stirring. Lisuride (5%) was then dissolved in acetone and added to the polymer mixture. Butylhydroxytoluene (1%), polyvidone 25 (10%), 1-dodecanol (10%), adhesive force booster (2%) and Aloe Vera oil (5%) were weighed out and added to the polymer solution while stirring. The polymer mixture was applied to a siliconised polyester film and then dried under the controlled action of moderate heat and laminated with polyester film in a continuously operating installation to give a dry lisuride laminate of 50 g/cm². The finished laminate was rolled into rolls. The laminate was stored as an intermediate product in polyethylene film (PE film) until processed further. The final production of the TTS was carried out in a punching and packing machine using a multistep process. At the first station, a withdrawable layer was cut into the laminate through the release layer, without cutting through the adhesive layer. The laminate was cut around the individual TTS' in the longitudinal and transverse direction. The TTS was then transferred by a suction head. A subsequent heat-sealing tool sealed the films in bags, each containing a TTS.

### DESCRIPTION OF THE FIGURES

FIG. 1a: Permeation profile of lisuride through excised hairless mouse skin, using acrylate-based trans-dermal systems without Aloe Vera oil and without 1-dodecanol. The mean (SD) of n=3 experiments is shown.

FIG. 1b: Permeation profile of lisuride through excised hairless mouse skin, using acrylate-based trans-dermal active-substance release systems containing 1-dodecanol but without Aloe Vera oil. The mean (SD) of n=9 experiments is shown. The regression lines of the linear range were incorporated in the diagram by a dashed line to graphically illustrate the lag time.

FIG. 1c: Permeation profile of lisuride through excised hairless mouse skin, using acrylate-based trans-dermal active-substance release systems containing Aloe Vera oil but without 1-dodecanol. The mean (SD) of n=3 experiments is shown. The regression lines of the linear range were incorporated in the diagram by a dashed line to graphically illustrate the lag time.

FIG. 2: Plasma concentrations time profile following application of lisuride plasters to elderly probands for 48 hours (the values shown are mean values).

FIG. 3: Simulation of lisuride plasma levels following transdermal application.

FIG. 4: Primary efficacy end point.

FIG. 5: In vitro release of lisuride from an oral immediate-release tablet preparation in water.

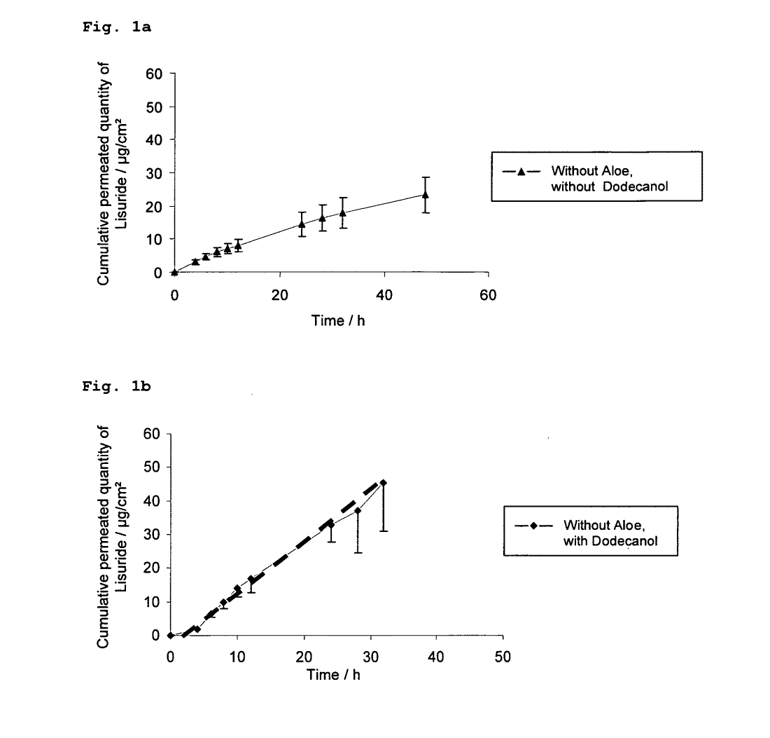

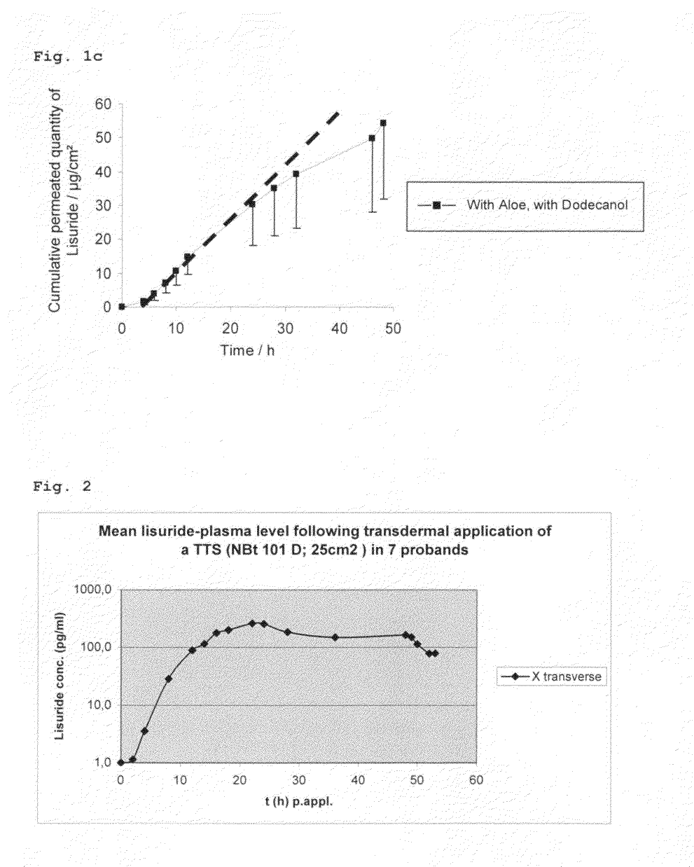

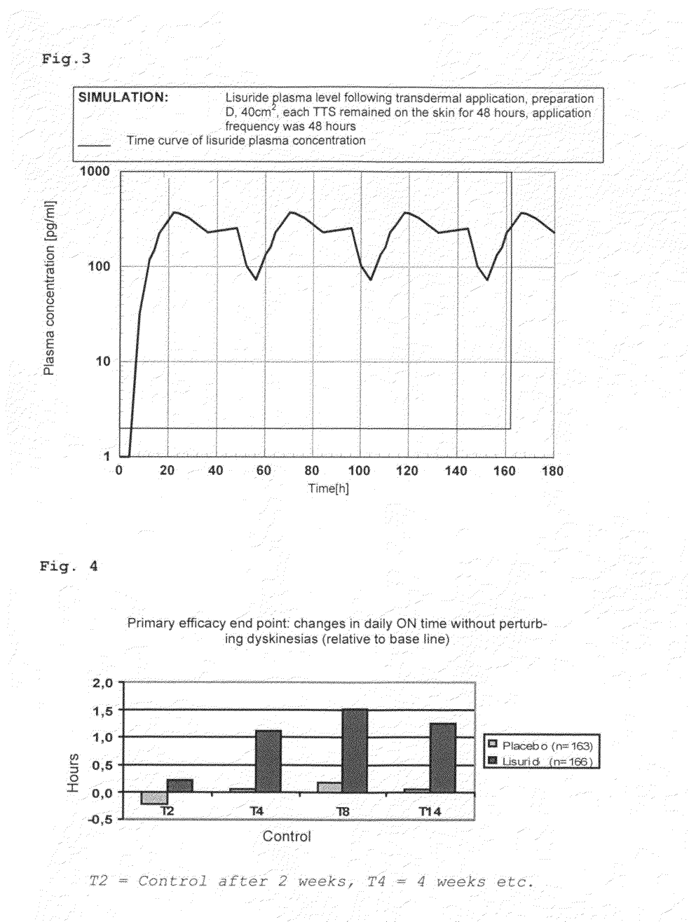

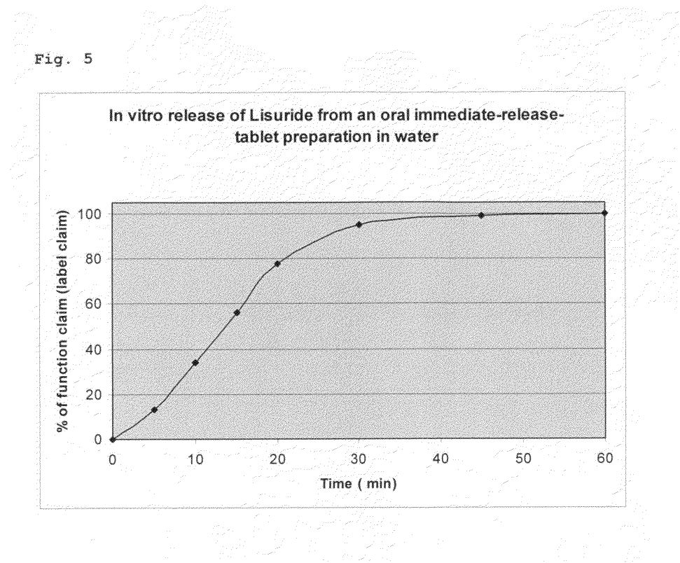

1. A transdermal therapeutic system (TTS) consisting of an impermeable coating, a matrix containing an ergoline compound having the 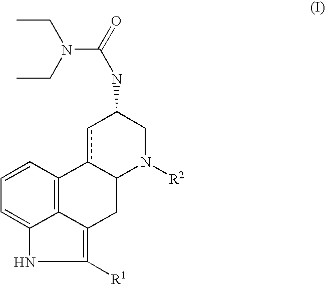
formula (I)
or a physiologically compatible salt thereof,
wherein R¹ denotes an H atom or a halogen atom and R² is an alkyl group having 1 to 4 carbon atoms and ----- denotes a single or double bond, and a removable protective layer, wherein the ergoline compound or a physiologically compatible salt thereof is stabilised by an antioxidant and a basic polymer, characterised in that the matrix contains at least one hydrocarbon having 8 to 18 carbon atoms in a straight or branched chain, which has a functional group at the end of the alkyl chain and/or Aloe Vera.

2. The transdermal therapeutic system (TTS) according to claim 1, characterised in that the at least one hydrocarbon having 8 to 18 carbon atoms in a straight or branched chain at the end of the alkyl group has a hydroxyl or amino group or a pyrrolidone ring or a —OOCCH₂N(CH₃)₂ group as a polar functional group.

3. The transdermal therapeutic system (TTS) according to claim 1, characterised in that the at least one hydrocarbon having 8 to 18 carbon atoms in a straight or branched chain has at the end of the alkyl group a hydroxyl group as a polar functional group.

4. The transdermal therapeutic system (TTS) according to claim 1, characterised in that the at least one hydrocarbon having a functional group at the end of the alkyl chain has 10 to 14 carbon atoms in a straight or branched chain.

5. The transdermal therapeutic system (TTS) according to claim 1, characterised in that the at least one hydrocarbon having a functional group at the end of the alkyl chain has 12 carbon atoms in a straight or branched chain.

6. The transdermal therapeutic system (TTS) according to claim 1, characterised in that the at least one hydrocarbon having a functional group at the end of the alkyl chain is 1-dodecanol.

7. The transdermal therapeutic system (TTS) according to claim 1, characterised in that the at least one hydrocarbon having a functional group at the end of the alkyl chain has a content of 0.001 to 20.00 wt. %.

8. The transdermal therapeutic system (TTS) according to claim 1, characterised in that the at least one hydrocarbon having a functional group at the end of the alkyl chain has a content of 0.50 to 15.00 wt. %.

9. The transdermal therapeutic system (TTS) according to claim 1, characterised in that the at least one hydrocarbon having a functional group at the end of the alkyl chain has a content of 1.00 to 10.00 wt. %.

10. The transdermal therapeutic system (TTS) according to claim 1, characterised in that the at least one hydrocarbon having a functional group at the end of the alkyl chain has a content of 10.00 wt. %.

11. The transdermal therapeutic system (TTS) according to claim 1, characterised in that the Aloe Vera oil was obtained from a vegetable fatty oil.

12. The transdermal therapeutic system (TTS) according to claim 1, characterised in that the Aloe Vera oil was obtained from peanut oil, almond oil, sesame oil or soya oil.

13. The transdermal therapeutic system (TTS) according to claim 1, characterised in that the Aloe Vera oil was obtained from soya oil.

14. The transdermal therapeutic system (TTS) according to claim 1, characterised in that the content of Aloe Vera oil is 0.01 to 20.00 wt. %.

15. The transdermal therapeutic system (TTS) according to claim 1, characterised in that the content of Aloe Vera oil is 0.5 to 10.00 wt. %.

16. The transdermal therapeutic system (TTS) according to claim 1, characterised in that the content of Aloe Vera oil is 5.00 wt. %.

17. The transdermal therapeutic system (TTS) according to claim 1, characterised in that the ergoline compound is lisuride or proterguride or a physiologically compatible salt thereof.

18. The transdermal therapeutic system (TTS) according to claim 1, characterised in that the ergoline compound is lisuride or proterguride.

19. The transdermal therapeutic system (TTS) according to claim 1, characterised in that the content of the ergoline compound or the physiologically compatible salt thereof is 0.50 to 20.00 wt. %.

20. The transdermal therapeutic system (TTS) according to claim 1, characterised in that the content of the ergoline compound or the physiologically compatible salt thereof is 3.00 to 6.00 wt. %.

21. The transdermal therapeutic system (TTS) according to claim 1, characterised in that the matrix contains penetration-boosting means.

22. The transdermal therapeutic system (TTS) according to claim 1, characterised in that the matrix has a covering diffusion barrier and an adhesive layer which is permeable to the substance according to claim 1.

23. The transdermal therapeutic system (TTS) according to claim 1, characterised in that the antioxidant is selected from the group of di-tent. butyl methyl phenols, tert. butyl methoxyphenols, tocopherols and/or ubiquinones.

24. The transdermal therapeutic system (TTS) according to claim 1, characterised in that the antioxidant is contained in quantities of 0.25 wt. % to 5.00 wt. %.

25. The transdermal therapeutic system (TTS) according to claim 1, characterised in that the basic polymer is an acrylate (co)polymer.

26. The transdermal therapeutic system (TTS) according to claim 24, characterised in that the acrylate (co)polymer is a butylmethacrylate-(2-diaminoethyl)methacrylate-methacrylate copolymer.

27. The transdermal therapeutic system (TTS) according to claim 22, characterised in that the basic polymer is contained in the matrix or the adhesive layer.

28. The transdermal therapeutic system (TTS) according to claim 27, characterised in that the basic polymer in the matrix or the adhesive layer contains an adhesive force booster.

29. The transdermal therapeutic system (TTS) according to claim 28, characterised in that the adhesive force booster contains resins and/or neutral polyacrylates.

30. The transdermal therapeutic system (TTS) according to claim 28, characterised in that the content of adhesive force booster is 1 to 20 wt. %.

31. The transdermal therapeutic system (TTS) according to claim 28, characterised in that the content of adhesive force booster is 2 to 10 wt. %.

32. A transdermal therapeutic system (TTS) according to claim 1 for the treatment of neurodegenerative diseases, wherein the TTS consists of an impermeable coating, a matrix containing an ergoline compound having the formula (I) 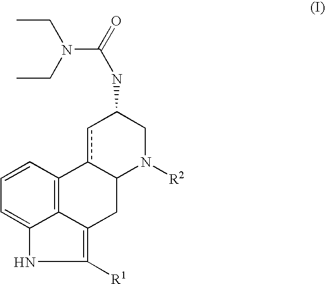
or a physiologically compatible salt thereof,
wherein R¹ denotes an H atom or a halogen atom and R² is an alkyl group having 1 to 4 carbon atoms and [symbol]
denotes a single or double bond, and a removable protective layer, wherein the ergoline compound or a physiologically compatible salt thereof is stabilised by an antioxidant and a basic polymer, characterised in that the matrix contains at least one hydrocarbon having 8 to 18 carbon atoms in a straight or branched chain, which has a functional group at the end of the alkyl chain and/or Aloe Vera.

33. A transdermal therapeutic system according to claim 1 for the treatment of Parkinson's disease and Parkinsonism.

34. A transdermal therapeutic system according to claim 1 for the treatment of restless legs syndrome.
